(19) United States
(12) Patent Application Publication
(10) Pub. No.: US 2022/0000702 A1
(43) Pub. Date: Jan. 6, 2022

(54) Pneumatic distributor and walking aid system including the same

(71) Applicant: KOREA INSTITUTE OF ROBOT AND CONVERGENCE, Pohang-si (KR)

(72) Inventors: Kap-Ho Seo, Pohang-si (KR); Jong-II Lee, Pohang-si (KR); Jaewan Koo, Pohang-si (KR); Kyon-Mo Yang, Pohang-si (KR); Donggi Gwak, Pohang-si (KR); Jaeyoul Lee, Pohang-si (KR); Dong-Seop Sohn, Pohang-si (KR)

(21) Appl. No.: 17/360,209

(22) Filed: Jun. 28, 2021

(30) Foreign Application Priority Data
Jul. 3, 2020 (KR) 10-2020-0082395

(51) Int. Cl. A61H 3/00; A61H 1/02

(52) U.S. Cl. CPC A61H 3/00; A61H 1/024; A61H 2201/1238; A61H 2201/165; A61H 2201/1642; A61H 2003/007; A61H 2201/1671; A61H 2201/5058

(57) ABSTRACT

Provided is a pneumatic distributor for intaking and exhausting air of an air pad, including a lower plate member having one side to which an intake port and an exhaust port are connected and the other side to which a first port and a second port connected to the air pad are connected, and an upper plate member rotatably coupled to an upper surface of the lower plate member and configured to relatively rotate to control intaking and exhausting air through the first port and the second port, wherein the lower plate member includes an intake recess communicating with the intake port, an exhaust recess communicating with the exhaust port, a first recess communicating with the first port, and a second recess communicating with the second port, and the intake recess, the exhaust recess, the first recess, and the second recess are formed on an upper surface of the lower plate member.

### CROSS REFERENCE TO RELATED APPLICATIONS

This application claims priority to Korean Patent Application No. 10-2020-0082395 filed on Jul. 3, 2020, the disclosure of which is incorporated by reference herein in its entirety.

### BACKGROUND OF THE DISCLOSURE

### Field of the Disclosure

The present disclosure relates to a pneumatic distributor connected to an air pad and a walking aid system including the same, and more particularly, to a pneumatic distributor capable of intaking or exhausting air by rotating an upper plate member with respect to a lower plate member and a simplified periodic walking aid system capable of adjusting a repeated intake and exhaust time points in a garment-type soft wearable suit gait that may improve a wearer's walking condition by a rotary pneumatic distributor.

### Related Art

Walking aid soft wearable suits, bedsore prevention mattresses, and massagers achieve a purpose of equipment and devices through repeated intaking/exhausting air and maintenance at an appropriate time on the built-in air pad. However, in the case of intake/exhaust control using the existing pneumatic solenoid valve, a software algorithm for control has to be additionally designed, and the number of available uses is limited according to a limitation of a driving mechanism. In the case of a device with a large number of air pads, a plurality of pneumatic solenoid valves should be applied according to a required number of intake and exhaust valves, and it is necessary to develop a control algorithm for the solenoid valve according to a state of each air pad. Therefore, an increase in the number of air pads may increase the number of solenoid valves for controlling pneumatic pressure and increase complexity of the control algorithm.

Periodically managing air inside the air pad using the pneumatic solenoid valve is a universal technology and has been conducted and applied in many areas. However, improvement has focused on a reduction in a size of valves and software performance for controlling a plurality of valves, rather than intake/exhaust integration of a distributor and periodic hardware control based on integration and rotation of intake/exhaust of the distributor. Also, there has never been an example of independently designing, implementing, and applying upper and lower plates to change an intake/exhaust cycle control.

Efforts have been made to improve performance of devices and equipment such as walking aid and rehabilitation through pneumatic control of air pads, and related technologies may be considered to be generalized technologies. However, improvement of performance of the control algorithm of the pneumatic solenoid valve may not secure efficiency in an environment in which the pneumatic control system having a limited size is required. Thus, it is necessary to secure the efficiency through diversification of the solenoid valves for each control cycle, but it takes a lot of time to improve the control algorithm and to miniaturize valve hardware.

### RELATED ART DOCUMENT

### Patent Document

(Patent document 1) Korean Patent Registration No. 10-0616636

### SUMMARY

The present disclosure provides a pneumatic distributor capable of intaking and exhausting air and controlling an intake/exhaust time point through periodic rotation of a lower plate portion, thereby miniaturizing an air pad-based device and equipment and reducing complexity of a control algorithm.

In an aspect, a pneumatic distributor for intaking and exhausting air of an air pad includes: a lower plate member having one side to which an intake port and an exhaust port are connected and the other side to which a first port and a second port connected to the air pad are connected; and an upper plate member rotatably coupled to an upper surface of the lower plate member and configured to relatively rotate to control intaking and exhausting air through the first port and the second port, wherein the lower plate member includes an intake recess communicating with the intake port, an exhaust recess communicating with the exhaust port, a first recess communicating with the first port, and a second recess communicating with the second port, and the intake recess, the exhaust recess, the first recess, and the second recess are formed on an upper surface of the lower plate member.

The upper plate member may include an intake recess formed in a predetermined angle range from a surrounding of a central portion of a lower surface of the upper plate member to one side in a radial direction and an exhaust hole formed in a predetermined angle range from an outer side of the central portion to the other side in the radial direction.

The intake recess may be formed at 360 degrees from the upper surface around a center hole formed in a penetrating manner at the center.

The exhaust recess of the lower plate member may include a circular recess portion formed at 360 degrees at an edge of the upper surface of the lower plate, a pair of inner recess portions formed on an inner side in the circular recess portion, and an outer communication recess portion formed at a predetermined angle outward in the circular recess portion and communicating with the exhaust port.

The pair of inner recess portions may each be formed at an angle of 10 to 20 degrees, and the outer communication recess portion may be formed at an angle of 40 to 50 degrees.

The first recess and the second recess of the lower plate member may be disposed to be spaced apart from the pair of inner recess portions by an interval of 90 degrees and face each other.

The first recess and the second recess may be formed at an angle of 10 to 20 degrees.

The intake recess of the upper plate member may include a circular recess portion formed at 360 degrees on a lower surface a center hole formed in a penetrating manner at the center, an arc-shaped recess portion formed at a predetermined angle on an outer side of the circular recess portion, a first connection recess portion formed to be connected between the circular recess portion and the arc-shaped recess portion, and a second connection recess portion formed on an outer side of the arc-shaped recess portion in the radial direction.

The arc-shaped recess portion may be formed at an angle of 110 to 140 degrees.

The exhaust recess of the upper plate member may be formed at an angle of 110 to 140 degrees.

In another aspect, a walking aid system includes: a plurality of soft actuators attached to a portion of a joint of a leg to support muscular strength of the leg during walking; a pressure generator injecting or discharging air to or from the plurality of soft actuators; and the pneumatic distributor connected between the pressure generator and the plurality of soft actuators and adjusting pressure of air.

The plurality of soft actuators may be disposed in an up-down direction on the rear of a knee.

The walking aid system may further include: a leg support module attached to upper and lower portions of the joint of the leg to perform a musculoskeletal role.

The leg support module may be disposed on an inner side and an outer side of the leg.

The walking aid system may further include a fixing band surrounding the plurality of soft actuators and the leg support module to attach the plurality of soft actuators and the leg support module to the leg.

The fixing band may be disposed above and below the knee.

### Advantageous Effects

According to the pneumatic distributor of the present disclosure, the single pneumatic distributor performs intaking and exhausting of air and controlling an intake/exhaust time point through periodic rotation of a lower plate portion, thereby miniaturizing an air pad-based device and equipment and reducing complexity of a control algorithm.

### BRIEF DESCRIPTION OF THE DRAWINGS

FIGS. 1A and 1B are a top perspective view and a bottom perspective view of a pneumatic distributor according to an embodiment of the present disclosure.

FIGS. 2A and 2B are top perspective views separately illustrating an upper plate member and a lower plate member in FIG. 1A.

FIGS. 3A and 3B are a bottom perspective view separately illustrating an upper plate member and a lower plate member in FIG. 1B.

FIGS. 4A and 4B are top perspective views illustrating a lower plate member cut and separated in a horizontal plane.

FIGS. 5A and 5B are bottom perspective views illustrating the lower plate member cut and separated in a horizontal plane.

FIGS. 6A and 6B are a top perspective view illustrating the upper plate member of FIG. 2A and a top perspective view illustrating the upper plate member cut and separated in a vertical plane.

FIGS. 7A and 7B are a bottom perspective view illustrating the upper plate member of FIG. 3A and a bottom perspective view illustrating the upper plate member cut and separated in a vertical plane.

FIG. 8A is a top perspective view of a lower plate member, and FIG. 8B is a bottom perspective view of an upper plate member.

FIG. 9A is a top view of a lower plate member and FIG. 9B is a bottom view of an upper plate member.

FIGS. 10A to 10C are plan views illustrating a positional relationship of an upper plate member with respect to a lower plate member performing intake, maintenance, and exhaust in a pneumatic distributor of the present disclosure.

FIGS. 11A to 11F are plan views illustrating a positional relationship of an upper plate member with respect to a lower plate member performing intake, maintenance, and exhaust in a pneumatic distributor of the present disclosure.

FIG. 12 is a partial perspective view illustrating that a walking aid system of the present disclosure is worn.

FIG. 13 is a partial front view, a side view, and a rear view illustrating that a garment type soft wearable suit is worn.

FIG. 14 is a conceptual diagram illustrating a configuration of an electric part of a walking aid system.

FIG. 15 is a perspective view illustrating an example of a pneumatic chamber.

FIG. 16 is a graph illustrating an evaluation result of pneumatic performance of a soft actuator according to the presence or absence of a pneumatic chamber.

FIG. 17 is a graph illustrating a current consumption evaluation result for operating a pressure generator according to the presence or absence of a pneumatic chamber.

### DESCRIPTION OF EXEMPLARY EMBODIMENTS

As the inventive concept allows for various changes and numerous embodiments, particular embodiments will be illustrated in the drawings and described in detail in the written description. However, this is not intended to limit the inventive concept to particular modes of practice, and it is to be appreciated that all changes, equivalents, and substitutes that do not depart from the scope of the inventive concept are encompassed in the inventive concept.

The terms used in the application are used to describe specific embodiments only and are not intended to limit the present disclosure. A singular expression includes a plural expression as long as they are clearly distinguished in the context. In the application, it should be understood that the terms such as “comprising”, “including” are intended to express that features, numbers, steps, operations, constituent elements, part, or combinations thereof described in the specification are present and do not exclude existence or additions of one or more other features, numbers, steps, operations, constituent elements, part, or combinations thereof.

Hereinafter, preferred embodiments of the present invention will be described in detail with reference to the accompanying drawings. In this case, in the drawings, the same components are denoted by the same reference symbols as possible. Further, the detailed description of well-known functions and constructions which may obscure the gist of the present invention will be omitted. For the same reason, some of the elements in the accompanying drawings are exaggerated, omitted, or schematically illustrated.

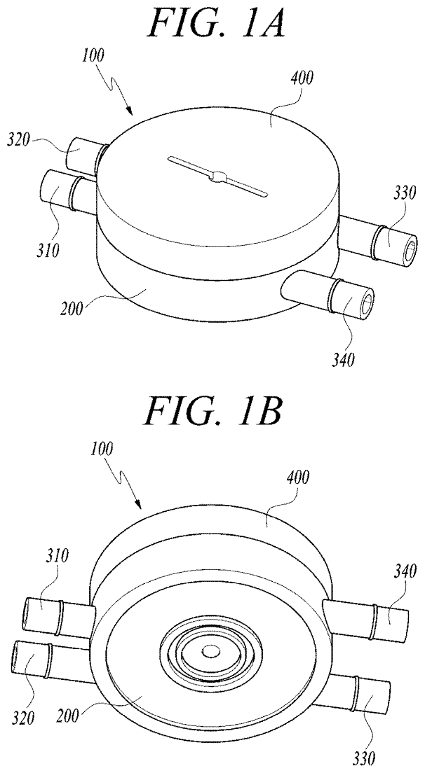

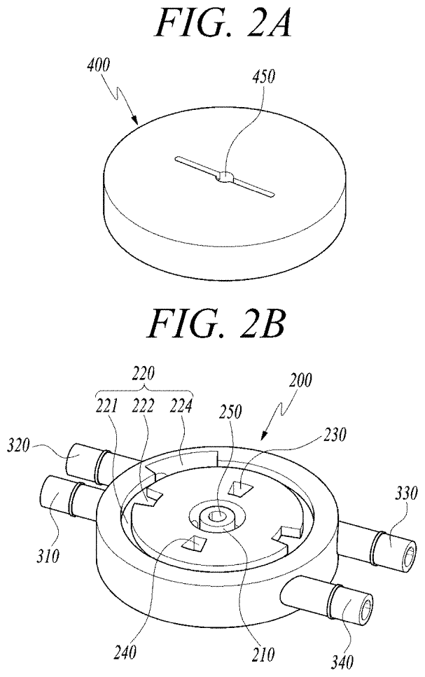

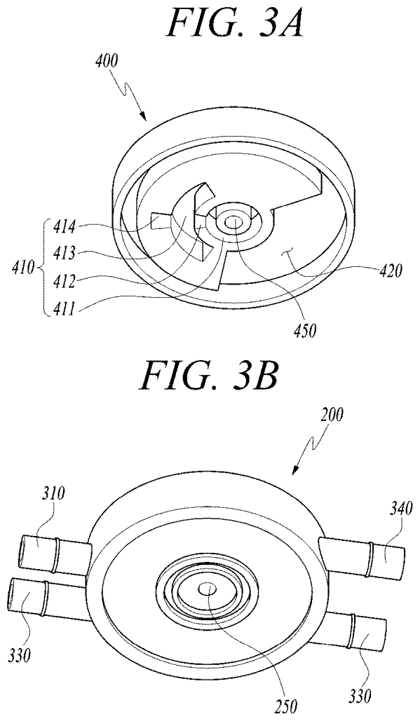

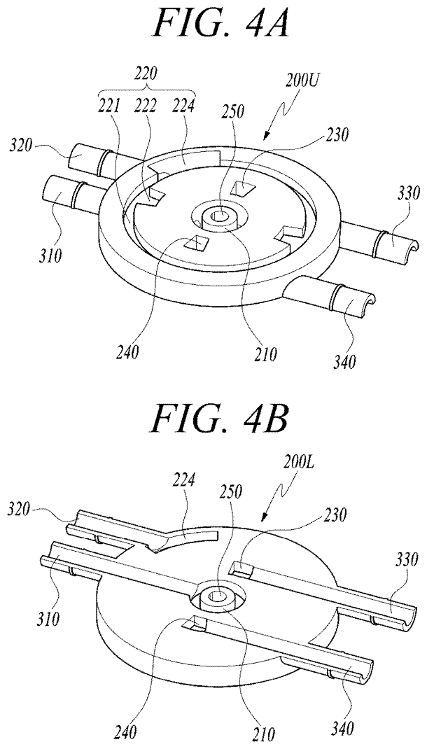

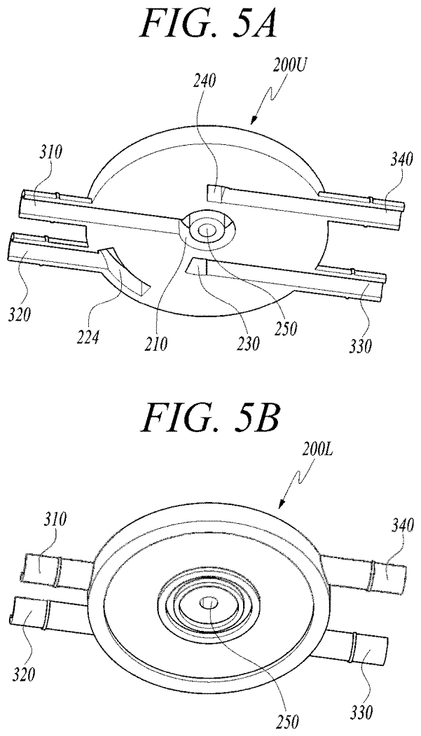

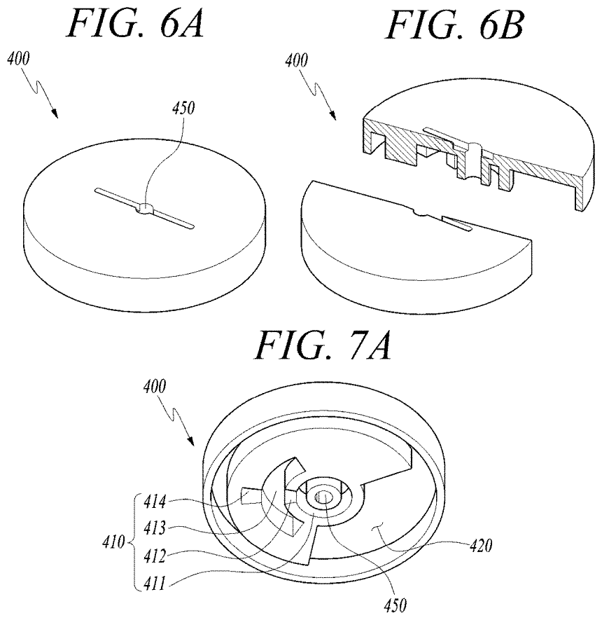

FIGS. 1A and 1B are a top perspective view and a bottom perspective view illustrating a pneumatic distributor according to an embodiment of the present disclosure, FIGS. 2A and 2B are top perspective views separately illustrating an upper plate member and a lower plate member in FIG. 1A. FIGS. 3A and 3B are a bottom perspective view separately illustrating an upper plate member and a lower plate member in FIG. 1B, FIGS. 4A and 4B are top perspective views illustrating a lower plate member cut and separated in a horizontal plane, FIGS. 5A and 5B are bottom perspective views illustrating the lower plate member cut and separated in a horizontal plane, and FIGS. 6A and 6B are a top perspective view illustrating the upper plate member of FIG. 2A and a top perspective view illustrating the upper plate member cut and separated in a vertical plane.

A pneumatic distributor 100 according to an embodiment of the present disclosure is for intaking and exhausting of air of an air pad (not shown), and includes a lower plate member 200 and an upper plate member 400. The lower plate member 200 and the upper plate member 400 are have a circular disk shape having a predetermined thickness, and air flow paths are formed on inner sides thereof facing each other.

As shown in FIGS. 1B to 3B, an intake port 310 and an exhaust port 320 may be connected to one side of the lower plate member 200, and a first port 330 and a second port 340 may be connected to the other side of the lower plate 200. That is, in the drawings, the intake port 310 and the exhaust port 320 may be connected to the left side of the lower plate member 200, and the first port 330 and the second port 340 may be connected to the right side of the lower plate member 200.

The first port 330 and the second port 340 may each be connected to the air pad by a connecting hose (not shown), and the intake port 310 and the exhaust port 320 may be open in the air or an air pump (not shown) may be connected thereto. The intake port 310 and the exhaust port 320 may be arranged parallel to each other, and may be arranged to be narrower than an interval between the first port 330 and the second port 340 arranged parallel to each other.

As the upper plate member 400 may be rotatably coupled to an upper surface of the lower plate member 200 and rotate relatively to control intake or exhaust through the first port 330 and the second port 340.

As shown in FIGS. 2B, 4A, 4B and 5A, the lower plate member 200 may include an intake recess 210 communicating with the intake port 310, an exhaust recess 220 communicating with the exhaust port 320, a first recess 230 communicating with the first port 330, and a second recess 240 communicating with the second port 340. The intake recess 320, the exhaust recess 220, the first recess 230, and the second recess 240 may be formed on an upper surface of the lower plate member 230.

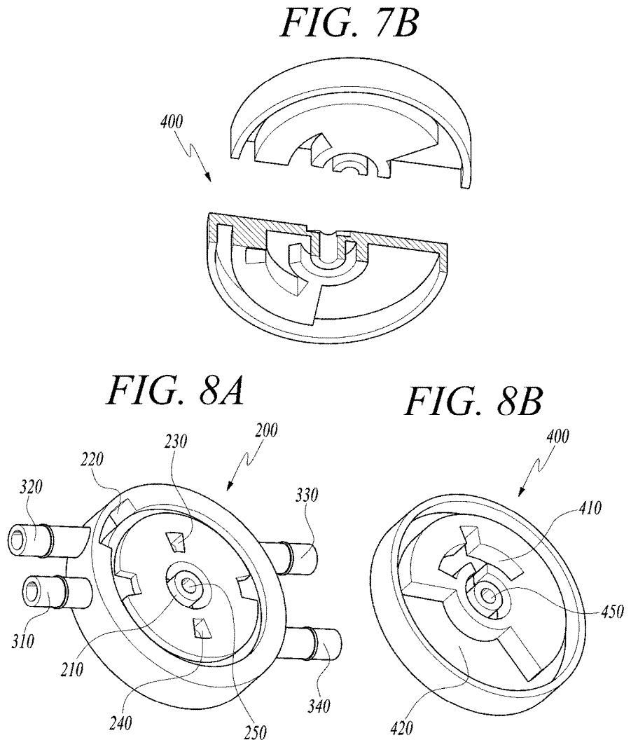

As shown in FIGS. 3A and 7A, the upper plate member 400 may include an intake recess 410 formed in a predetermined angle range from a surrounding of a central portion of a lower surface to one side in a radial direction and an exhaust recess 420 formed in a predetermined angle range from an outer side of the central portion to the other side in the radial direction.

As shown in FIGS. 2B and 3B, the lower plate member 200 may include a center hole 250 formed in a penetrating manner vertically at the center, and may include a center hole 450 formed in a penetrating manner vertically at the center. A rotational shaft (not shown) may be inserted into the center hole 250 of the lower plate member 200 and the center hole 450 of the upper plate member 400 to relatively rotate the upper plate member 400 with respect to the lower plate member 200. The upper plate member 400 may be rotated manually or may be automatically rotated by installing a motor. A linear recess passing through the center hole 450 may be formed on the upper surface of the upper plate member 400, and the upper plate member 400 may be manually or automatically rotated by inserting a tool such as a straight drive into the linear recess. The upper plate member 400 may be rotated in one direction according to a predetermined period, or may be rotated forward and reverse to a position in a predetermined period.

As shown in FIGS. 4A and 4B, the intake recess 210 of the lower plate member 200 may be formed at 360 degrees from the upper surface around the center hole 250 formed at the center in a penetrating manner. As shown in FIGS. 4A to 5B, the intake recess 210 may be formed as a concentric ring-shaped recess around the center hole 250, and may be connected to communicate with the intake port 310 on an outer surface thereof. The intake recess 210 may be formed to have a depth of ¾ or more of a thickness of the lower plate member 200.

As shown in FIG. 4A, the exhaust recess 220 of the lower plate member 200 may include a circular recess portion 221 formed at 360 degrees at an edge of the upper surface of the lower plate member, a pair of inner recess portions 222 formed on an inner side in the circular recess portion, and an outer communication recess portion 224 formed at a predetermined angle outward in the circular recess portion and communicating with the exhaust port 320.

The circular recess portion 221 may be formed to have a shallow depth in the form of a ring of 360 degrees on the edge of the upper surface of the lower plate member 200, for example, to have a depth less than ¼ of the thickness of the lower plate member 200. The pair of inner recess portions 222 may be formed inwardly from an inner circumferential surface of the circular recess portion 221, that is, in the center direction. The pair of inner recess portions 222 may be formed to have the same depth as the circular recess portion 221. The outer communication recess portion 224 may be formed to have a depth of three times or more of the circular recess portion 221, for example, a depth of ¾ or more of the thickness of the lower plate member 200, in a position in which an extension line of the exhaust port 320 meets the outer communication recess portion 224. The exhaust port 320 may be connected to an outer circumferential surface of the outer communication recess portion 224 to communicate with one end portion in a circumferential direction.

A pair of inner recess portions 222 may be formed at an angle of 10 to 20 degrees, and the outer communication recess portion 224 may be formed at an angle of 40 to 50 degrees. The formation position and angle of the pair of inner recess portions 222 and the outer communication recess portion 224 may determine an intake or exhaust period of air.

The first recess 230 and the second recess 240 of the lower plate member 200 may be disposed to face each other at an interval of 90 degrees with a pair of inner recess portions. The first recess 230 may be disposed at a position through which an extension line of the intake port 310 passes, and the second recess 240 may be disposed on the opposite side of the center hole 250 through which the extension line of the intake port 310 passes.

The first recess 230 and the second recess 240 may be formed at an angle of 10 to 20 degrees, respectively. The first recess 230 may be connected to communicate with the first port 330, and the second recess 240 may be connected to communicate with the second port 240.

The intake recess 410 of the upper plate member 400 may include a circular recess portion 411 formed at 360 degrees on a lower surface a central hole 450 formed in a penetrating manner at the center, an arc-shaped recess portion 413 formed at a predetermined angle on an outer side of the circular recess portion 411, a first connection recess portion 412 formed to be connected between the circular recess portion 411 and the arc-shaped recess portion 413, and a second connection recess portion 414 formed on an outer side of the arc-shaped recess portion 413 in the radial direction.

The circular recess portion 411 of the upper plate member 400 may be formed in the same shape and size as the circular recess portion 221 of the lower plate member 200. Therefore, the circular recess portion 411 of the upper plate member 400 may always overlap and communicate with the circular recess portion 221 of the lower plate member 200 regardless of rotational position of the lower plate member 200.

The arc-shaped recess portion 413 may be formed to be spaced apart from an outer side of the circular recess portion 411 in a radial direction and may be formed at an angle of 110 to 140 degrees. The arc-shaped recess portion 413 of the upper plate member 400 may be selectively formed in a radial position overlapping the first recess 230 or the second recess 240 of the lower plate member 200 according to rotational position thereof.

When the arc-shaped recess portion 413 of the upper plate member 400 is in a rotational position overlapping the first recess 230 of the lower plate member 200, air may be intaken from the intake port 310 to the first port 330. In addition, when the arc-shaped recess portion 413 of the upper plate member 400 is in a rotational position overlapping the second recess 240 of the lower plate member 200, air may be intaken from the intake port 310 to the second port 340.

The first connection recess portion 412 may be formed to be connected between the circular recess portion 411 and the arc-shaped recess portion 413, and when the arc-shaped recess portion 413 of the upper plate member 400 is in the rotational position overlapping the first recess or the second recess 240 of the lower plate member 200, the first connection recess portion 412 may allow air introduced into the arc-shaped recess portion 413 to be introduced into the circular recess portion 411.

The second connection recess portion 414 may be formed on an outer side of the arc-shaped recess portion 413 in the radial direction and may be formed in a radial position overlapping one of the pair of inner recess portions 222 of the lower plate member 200 selectively according to a rotation position thereof. When the second connection recess portion 414 of the upper plate member 400 is in a rotational position overlapping one of the pair of inner recess portions 222 of the lower plate member 200, air introduced into the intake port 310 may flow out to the exhaust port 320 through the intake recess 410 of the upper plate member 400 and the exhaust recess 220 of the lower plate member 200.

The exhaust recess 420 of the upper plate member 400 may be spaced apart from an outer side of the circular recess portion 411 in the radial direction and may be formed at an angle of 110 to 140 degrees. The exhaust recess 420 may be formed of a fan-shaped recess having a predetermined depth, and an edge portion of the exhaust recess 420 may be formed in a 360-degree circular ring shape so as to always overlap the outer communication recess portion 224 of the lower plate member 200. When the exhaust recess 420 is in a rotational position overlapping the first recess 230, air from the first port 330 may be exhausted to the exhaust port 320, and the exhaust recess 420 is in a rotational position overlapping the second recess 240, air of the second port 340 may be exhausted to the exhaust port 320.

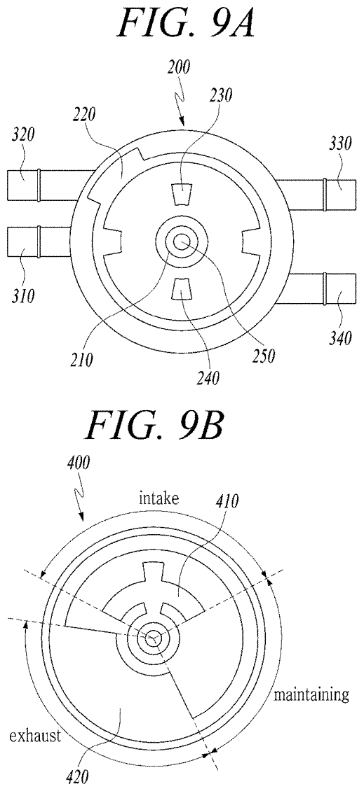

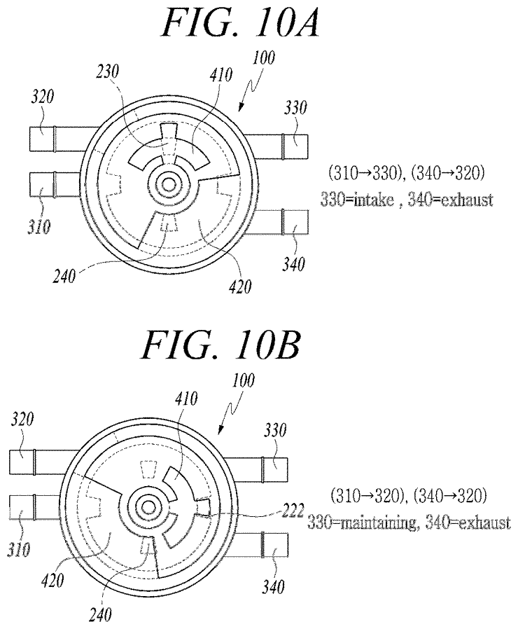

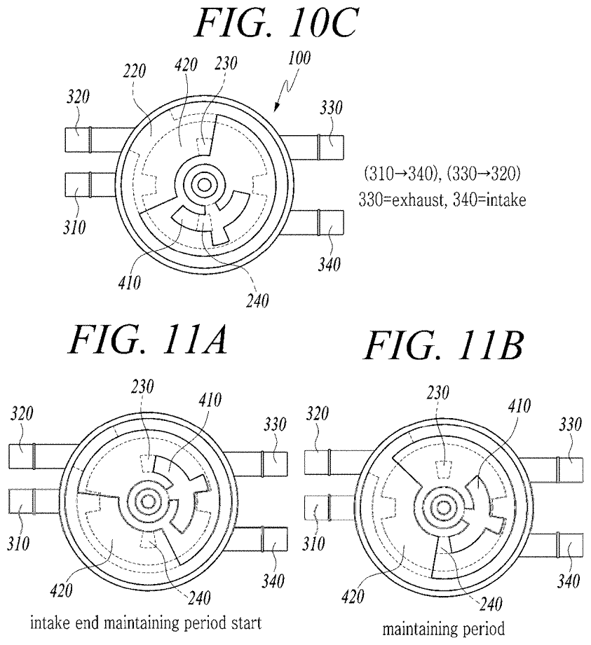

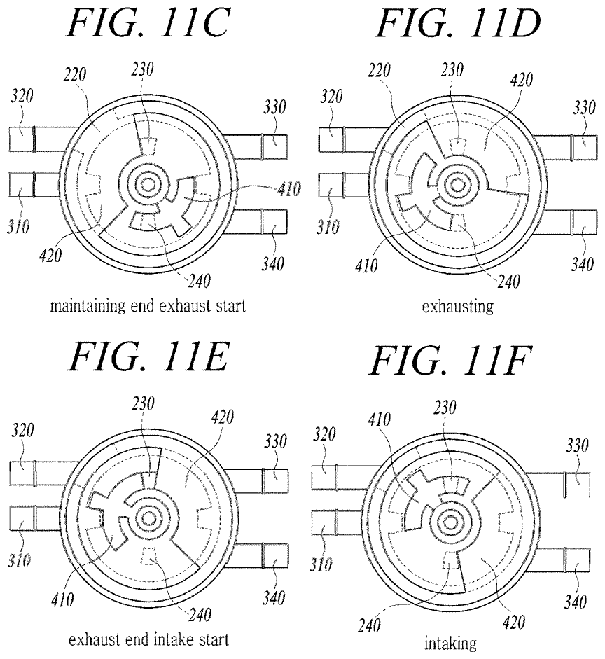

FIG. 8A is a top perspective view of a lower plate member, and FIG. 8B is a bottom perspective view of an upper plate member, FIG. 9A is a top view of a lower plate member and FIG. 9B is a bottom view of an upper plate member, FIGS. 10A to 10C are plan views illustrating a positional relationship of an upper plate member with respect to a lower plate member performing intake, maintenance, and exhaust in a pneumatic distributor of the present disclosure, and FIGS. 11A to 11F are plan views illustrating the positional relationship of an upper plate member with respect to a lower plate member performing intake, maintenance, and exhaust in a pneumatic distributor of the present disclosure.

Since the upper plate member 400 in FIGS. 8B and 9B is shown in a bottom perspective view and a bottom view, the upper plate member 400 shown in FIG. 7A is turned upside down. That is, it will be understood that the upper plate member 400 of FIGS. 8B and 9B is turned over and coupled to the lower plate member 200 by inserting a rotational shaft into the center holes 250 and 450.

As shown in FIG. 9B, a period in which air is intaken into the first port 330 or the second port 340 when the first recess 230 or the second recess 240 of the lower plate member 200 is located in the intake recess 410 of the upper plate member 400 is shown. In addition, a period in which air is exhausted from the first port 330 or the second port 340 when the first recess 230 or the second recess 240 of the lower plate member 200 is located in the exhaust recess 420 of the upper plate member 400 is shown. In FIG. 9B, a maintaining period is shown between the intake period and the exhaust period (right period), in which intaking or exhausting of air does not occur in the first port 330 or the second port 340.

FIGS. 10A to 10C show that recesses of the lower plate member 200 and the upper plate member 400 overlap each other, illustrating an air flow among the intake port 310, the exhaust port 320, the first port 330, and the second port 340 according to rotation of the upper plate member 400 of the distributor 100.

In FIG. 10A, since the intake recess 410 overlaps the first recess 230, air is intaken from the intake port 310 to the first port 330, and since the exhaust recess 420 overlaps the second recess 240, air is exhausted from the second port 340 to the exhaust port 320.

In the case of FIG. 10B, since the intake recess 410 overlaps the inner recess portion 222 of the exhaust recess 420, air flows from the intake port 310 to the exhaust port 320, and since the exhaust recess 420 overlaps the second recess 240, air is exhausted from the second port 340 to the exhaust port 320. Here, since neither the intake recess 410 nor the exhaust recess 420 overlap the first recess 230, the first port 330 is maintained without intaking or exhaust of air.

In the case of FIG. 10C, since the exhaust recess 420 overlaps the first recess 230, air is exhausted from the first port 330 to the exhaust port 320, and since the intake recess 410 overlaps the second recess 240, air is intaken from the intake port 310 to the second port 340.

When the upper plate member 400 is rotated 360 degrees with respect to the lower plate member 200, the first port 330 and the second port 340 may separately perform air intaking, maintaining, or air exhausting from or to the air pad.

FIGS. 11A to 11F show six rotational positions while the upper plate member 400 is mounted on the lower plate member 200 and rotates 360 degrees in a clockwise direction. Intaking, maintaining and or exhausting of air represent a function performed in terms of the first port 330.

In the case of FIG. 11A, since the intake recess 410 has just passed the first recess 230, the first port 330 indicates intake end and maintaining period start point. Here, since the exhaust recess 420 overlaps the second recess 240, the second port 340 is exhausting air.

In the case of FIG. 11B, the intake recess 410 is in a period before overlapping the second recess 240, and the first port 330 indicates maintaining period. Here, the second port 340 indicates exhaust end and maintain period start point.

In the case of FIG. 11C, since the exhaust recess 420 starts to overlap the first recess 230, the first port 330 indicates maintaining period end and exhaust start point. Here, since the intake recess 410 overlaps the second recess 240, the second port 340 is intaking air.

In the case of FIG. 11D, since the exhaust recess 420 overlaps the first recess 230, the first port 330 is exhausting air. Here, since the intake recess 410 has just passed the second recess 240, the second port 340 indicates intake end and maintaining period start point.

In the case of FIG. 11E, since the exhaust recess 420 has just passed the first recess 230 and the intake recess 410 starts to overlap the first recess 230, the first port 330 indicates exhaust end and intake start point. Here, the second port 340 is in the maintaining period.

In the case of FIG. 11F, since the intake recess 410 overlaps the first recess 230, the first port 330 is intaking air. Here, since the exhaust recess 420 starts to overlap the second recess 240, the second port 340 indicates maintaining period end and exhaust start point.

According to the pneumatic distributor of the present disclosure, by rotating the upper plate member with respect to the lower plate member and moving the upper plate member to a predetermined rotational position, intaking, exhausting, or maintaining air may be easily operated through the first port and the second port connected to the air pad.

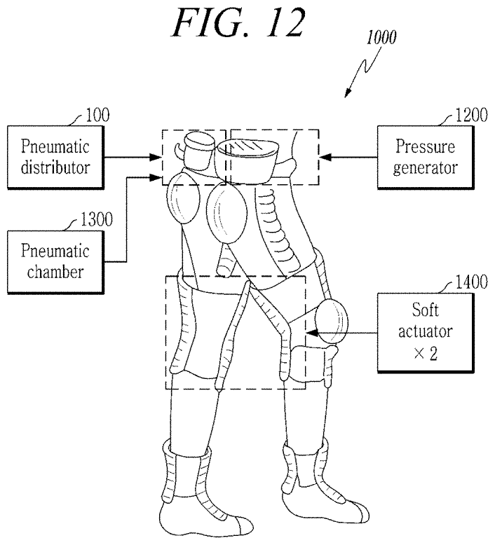

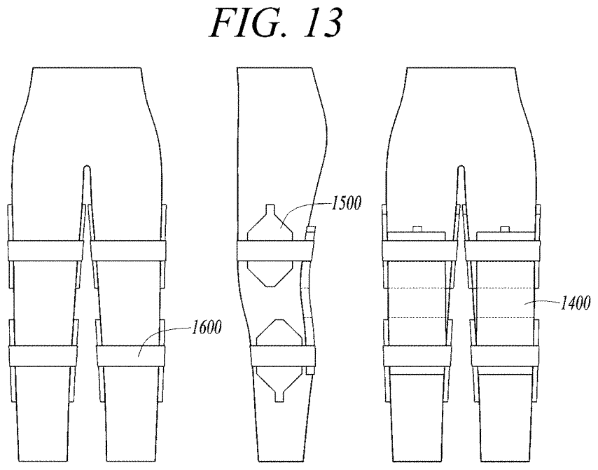

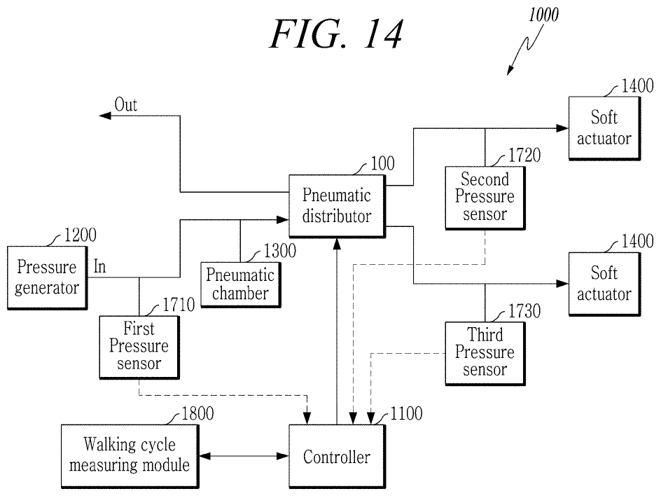

FIG. 12 is a partial perspective view illustrating that a walking aid system of the present disclosure is worn, FIG. 13 is a partial front view, a side view, and a rear view illustrating that a garment type soft wearable suit is worn, FIG. 14 is a conceptual diagram illustrating a configuration of an electric part of a walking aid system, and FIG. 15 is a perspective view illustrating an example of a pneumatic chamber.

The walking aid system 1000 of the present disclosure may include a plurality of soft actuators 1400 attached to a joint part of a leg to support muscle strength of the leg, a pressure generator 1200 injecting or discharging air to or from the plurality of soft actuators, and the aforementioned pneumatic distributor 100 connected between the pressure generator 1200 and the plurality of soft actuators 1400 to adjust air pressure.

The soft actuator 1400 is an air bag or air pad formed of a flexible material, and an external shape thereof may be changed by injecting or discharging air into or from a pocket thereof. The plurality of soft actuators 1400 may be attached to the back of the knee, that is, to be vertically disposed on the popliteal regions of the left and right legs to auxiliary support muscle strength of the legs when the wearer walks.

The pressure generator 1200 may generate and supply compressed air like an air compressor. The pressure generator 1200 may be worn on the wearer's waist.

As described above, the pneumatic distributor 100 may include the lower plate member 200 and the relatively rotating upper plate member 400, and the intake port 310, the exhaust port 320, the first port 330, and the second port 340 may be connected to the lower plate member 200. One pneumatic distributor 100 may replace the role of four solenoid valves in the existing walking aid system. In the pneumatic distributor 100, as the upper plate member 400 relatively rotates with respect to the lower plate member 200 at a predetermined speed, intaking or exhausting of air supplied from the pressure generator 1200 to the plurality of soft actuators 1400 or discharged therefrom may be periodically adjusted.

A plurality of air hoses may be connected among the pressure generator 1200, the pneumatic distributor 100, and the plurality of soft actuators 1400.

As shown in FIG. 13, the walking aid system 1000 of the present disclosure may include a leg support module 1500 attached to upper and lower portions of a leg joint to perform a musculoskeletal role and a fixing band 1600 surrounding the plurality of soft actuators 1400 and the leg support module 1500 to attach the plurality of soft actuators 1400 and the leg support module 1500 to the leg. The soft actuator 1400, the leg support module 1500, and the fixing band 1600 may constitute a garment-type soft wearable suit.

The leg support module 1500 may be disposed on inner and outer sides of the leg and attached to the upper and lower portions of the leg joint, thereby serving as a musculoskeletal support for parts other than the joint. Four leg support modules 1500 may be attached to the inner and outer sides of the leg above and below the knee in one leg.

The fixing band 1600 is a band surrounding and pressing the soft actuator 1400 and the leg support module 1500 to attach the soft actuator 1400 and the leg support module 1500 around the leg. The fixing band 1600 may be formed of an elastic material or provided with a Velcro tape to be easily detachable. In one leg, an upper fixing band 1600 may wrap and fix an upper portion of one soft actuator 1400 and two leg support modules 1500 above the knee, and a lower fixing band 1600 may wrap and fix a lower portion of one soft actuator 1400 and two leg support modules 1500 below the knee. That is, two fixing bands 1700 may be arranged above and below the knee of one leg.

As shown in FIGS. 12 and 14, the walking aid system 1000 of the present disclosure may further include a pneumatic chamber 1300 connected between the pressure generator 1200 and the pneumatic distributor 100 to temporarily store air pressure supplied from the pressure generator 1200 and supply the air pressure to the pneumatic distributor 100. The pneumatic chamber 1300 may be connected to a pneumatic flow path connected from the pressure generator 1200 to the intake port 310 of the pneumatic distributor 100.

As shown in FIG. 15, the pneumatic chamber 1300 may be formed as a corrugated tube to be deformed according to air pressure therein. For example, the pneumatic chamber 1300 may be formed in a substantially rectangular parallelepiped shape that is deformable from 100 mm in width, 50 mm in length, and 10 mm to 100 mm in height. One side of the pneumatic chamber 1300 may be provided with an intake hose and an exhaust hose connected to the pneumatic flow path. The pneumatic chamber 1300 may allow pneumatic pressure increased in a maintaining and exhaust stage of the pneumatic distributor 100 to be transferred to the soft actuator 1400 in an intake stage even if no external force is applied thereto.

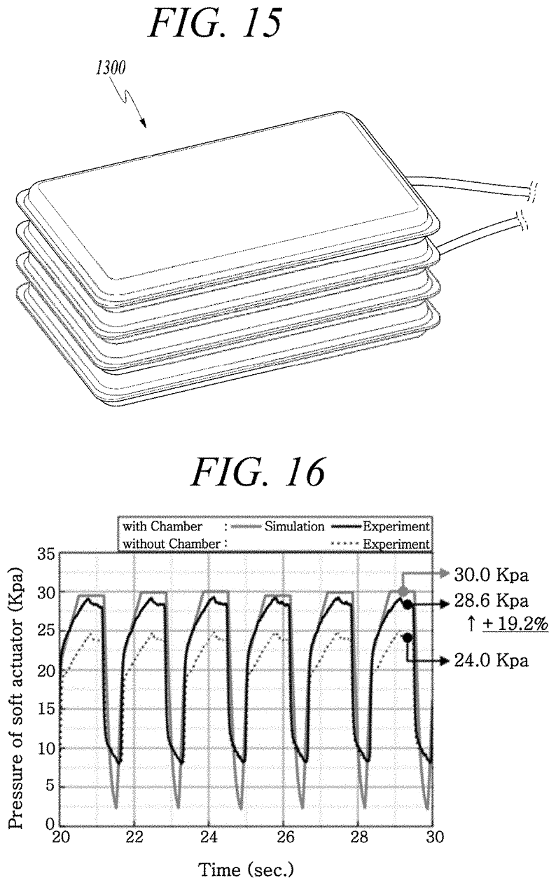

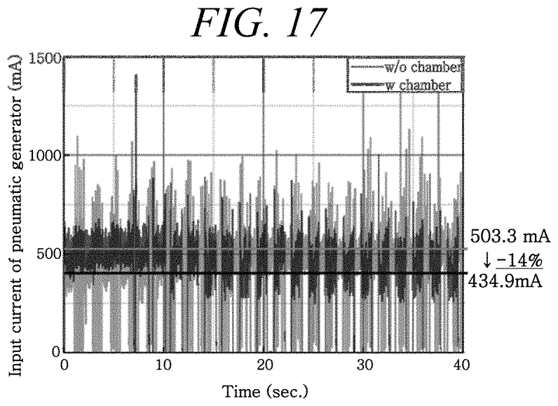

FIG. 16 is a graph illustrating an evaluation result of pneumatic performance of a soft actuator according to the presence or absence of a pneumatic chamber, and FIG. 17 is a graph illustrating a current consumption evaluation result for operating a pressure generator according to the presence or absence of a pneumatic chamber.

In order to determine performance of the soft actuator according to the presence or absence of a pneumatic chamber, an operation experiment of the walking aid system 1000 of the present disclosure was conducted.

As shown in FIG. 16, with the pneumatic chamber, a maximum pressure that may be applied to the soft actuator was 30.0 kPa as a result of simulation evaluation, and 28.6 kPa as a result of an actual experiment of the walking aid system. It can be seen that the maximum pressure was increased by about 19.2%, compared with a case in which the maximum pressure was 24.0 kPa without the pneumatic chamber.

As shown in FIG. 17, as a result of evaluation of current consumption for operation of the pressure generator with or without a pneumatic chamber, an average current consumption was 434.9 mA with the pneumatic chamber and 503.3 mA without the pneumatic chamber. Thus, it can be seen that, with the pneumatic chamber, the average current consumption of the pressure generator was reduced by about 14%, compared to the case without the pneumatic chamber.

As shown in FIG. 14, the walking aid system 1000 of the present disclosure may further include a gait cycle recognition module 1800 including a plurality of inertial measurement unit (IMU) sensors to recognize a gait cycle of a walker.

The gait cycle recognition module 1800 may be a device capable of acquiring biometric information such as a user's gait movement by mounting an IMU sensor in a soft wearable suit. The IMU sensors include 6-axis sensor including a gyroscope, and an accelerometer and a 9-axis sensor including a gyroscope, an accelerometer, and a geomagnetic sensor. A gyroscope may measure angular velocity (rad/s), i.e., how many degrees it rotates per unit time. An accelerometer may measure acceleration in x-axis, y-axis, and z-axis directions when an object moves in a three-dimensional space. A geomagnetic sensor (i.e., a magnetometer or compass) may compensate for an error of a gyroscope.

A total of five IMU sensors may be placed behind the wearer's waist, left and right thighs and calves. When the soft wearable suit is worn, a human body model may be constructed based on a movement of the wearer, and the wearer's posture and gait cycle may be estimated based on the movement of the human body model. Accordingly, if the wearer wears the soft wearable suit and walks for a predetermined period of time, the wearer's gait cycle may be acquired by the gait cycle recognition module 1800. A gait cycle signal measured by the gait cycle recognition module 1800 may be transmitted to the controller 1100, and the controller 1100 may control an operation (rotational speed) of the pneumatic distributor 100 according to the gait cycle of the wearer.

As shown in FIG. 14, a portion of high-pressure air generated by the pressure generator 1200 may be supplied to the pneumatic chamber 1300, and the rest may be selectively supplied to the plurality of soft actuators 1400 through the pneumatic distributor 10, and air inside the plurality of soft actuators 1400 may be discharged to the outside through the pneumatic distributor 100. Here, a first pressure sensor 1710 may be provided between the pressure generator 1200 and the pneumatic distributor 100 to detect pressure of air supplied to the pneumatic distributor 100. In addition, a second pressure sensor 1720 may be provided between the pneumatic distributor 100 and the soft actuator 1400 of the left leg to detect the pressure of the air supplied to the soft actuator 1400. In addition, a third pressure sensor 1730 may be provided between the pneumatic distributor 100 and the soft actuator 1400 of the right leg to detect pressure of the air supplied to the soft actuator 1400.

In this manner, the pressure sensors are provided between the pressure generator 1200 and the pneumatic distributor 100 and between the pneumatic distributor 100 and the soft actuator 1400 to measure pressure of the air, so that the walking aid system 1000 may feedback-control the pneumatic distributor 100 so that an appropriate air pressure may be applied according to the gait cycle when the wearer walks through the controller 1100. The controller 1100 may control a speed of rotating the pneumatic distributor 100 by an electric motor according to the gait cycle of the wearer measured by the gait cycle recognition module 1800, thereby supporting the wearer's posture when the wearer walks and supporting the walking motion.

Although the embodiments of the present invention have been described hereinabove, those having ordinary knowledge in the technical field of the present invention will appreciate that various changes and modifications may be made to the embodiments described herein by the addition, modification, removal and the like of elements without departing from the scope and spirit of the present invention as disclosed in the accompanying claims. However, the various changes and modifications are to be construed as being included within the right scope of the present invention.

What is claimed is:

1. A pneumatic distributor for intaking and exhausting air of an air pad, the pneumatic distributor comprising:
a lower plate member having one side to which an intake port and an exhaust port are connected and the other side to which a first port and a second port connected to the air pad are connected; and
an upper plate member rotatably coupled to an upper surface of the lower plate member and configured to relatively rotate to control intaking and exhausting air through the first port and the second port,
wherein the lower plate member includes an intake recess communicating with the intake port, an exhaust recess communicating with the exhaust port, a first recess communicating with the first port, and a second recess communicating with the second port, and the intake recess, the exhaust recess, the first recess, and the second recess are formed on an upper surface of the lower plate member.

2. The pneumatic distributor of claim 1, wherein the upper plate member includes an intake recess formed in a predetermined angle range from a surrounding of a central portion of a lower surface of the upper plate member to one side in a radial direction and an exhaust hole formed in a predetermined angle range from an outer side of the central portion to the other side in the radial direction.

3. The pneumatic distributor of claim 2, wherein the intake recess is formed at 360 degrees from the upper surface around a center hole formed in a penetrating manner at the center.

4. The pneumatic distributor of claim 3, wherein the exhaust recess of the lower plate member includes a circular recess portion formed at 360 degrees at an edge of the upper surface of the lower plate, a pair of inner recess portions formed on an inner side in the circular recess portion, and an outer communication recess portion formed at a predetermined angle outward in the circular recess portion and communicating with the exhaust port.

5. The pneumatic distributor of claim 4, wherein the pair of inner recess portions are each formed at an angle of 10 to 20 degrees, and the outer communication recess portion is formed at an angle of 40 to 50 degrees.

6. The pneumatic distributor of claim 4, wherein the first recess and the second recess of the lower plate member are disposed to be spaced apart from the pair of inner recess portions by an interval of 90 degrees and face each other.

7. The pneumatic distributor of claim 6, wherein the first recess and the second recess are formed at an angle of 10 to 20 degrees.

8. The pneumatic distributor of claim 2, wherein the intake recess of the upper plate member includes a circular recess portion formed at 360 degrees on a lower surface a center hole formed in a penetrating manner at the center, an arc-shaped recess portion formed at a predetermined angle on an outer side of the circular recess portion, a first connection recess portion formed to be connected between the circular recess portion and the arc-shaped recess portion, and a second connection recess portion formed on an outer side of the arc-shaped recess portion in the radial direction.

9. The pneumatic distributor of claim 8, wherein the arc-shaped recess portion is formed at an angle of 110 to 140 degrees.

10. The pneumatic distributor of claim 9, wherein the exhaust recess of the upper plate member is formed at an angle of 110 to 140 degrees.

11. A walking aid system comprising:
a plurality of soft actuators attached to a portion of a joint of a leg to support muscular strength of the leg during walking;
a pressure generator injecting or discharging air to or from the plurality of soft actuators; and
the pneumatic distributor connected between the pressure generator and the plurality of soft actuators and adjusting pressure of air according to claim 1.

12. The walking aid system of claim 11, further comprising: a pneumatic chamber connected between the pressure generator and the pneumatic distributor, temporarily storing pressure of air supplied from the pressure generator, and supplying the stored pressure of air to the pneumatic distributor.

13. The walking aid system of claim 12, further comprising:
a walking period recognition module including a plurality of inertial measurement unit (IMU) sensors to recognize a walking period of a walker.

14. The walking aid system of claim 12, wherein the plurality of soft actuators are disposed in an up-down direction on the rear of a knee.

15. The walking aid system of claim 12, further comprising:
a leg support module attached to upper and lower portions of the joint of the leg to perform a musculoskeletal role.

16. The walking aid system of claim 15, wherein the leg support module is disposed on an inner side and an outer side of the leg.

17. The walking aid system of claim 15, further comprising:
a fixing band surrounding the plurality of soft actuators and the leg support module to attach the plurality of soft actuators and the leg support module to the leg.

18. The walking aid system of claim 17, wherein the fixing band is disposed above and below the knee.
